(19) United States
(12) Patent Application Publication
(10) Pub. No.: US 2015/0098954 A1
(43) Pub. Date: Apr. 9, 2015

(54) Compositions and Methods Related to CRISPR Targeting

(71) Applicant: Elwha LLC, Bellevue, WA (US)

(72) Inventors: Roderick A. Hyde, Redmond, WA (US); Wayne R. Kindsvogel, Seattle, WA (US); Lowell L. Wood, Bellevue, WA (US)

(21) Appl. No.: 14/048,283

(22) Filed: Oct. 8, 2013

(51) Int. Cl. C12N 15/85

(52) U.S. Cl. CPC C12N 15/85
USPC 424/184.1; 435/320.1; 435/455; 514/44 R

(57) ABSTRACT

Disclosed herein include embodiments related to addition, deletion, or modification of DNA, RNA, or protein in a subject. In an embodiment, the DNA, RNA, or protein is endogenous. In an embodiment, the DNA, RNA, or protein is exogenous. Further embodiments relate to computerized systems for assisting in the disclosed methods.

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority Application(s)). In addition, the present application is related to the “Related Applications,” if any, listed below.

### PRIORITY APPLICATIONS

None.

### RELATED APPLICATIONS

None.

If the listings of applications provided above are inconsistent with the listings provided via an ADS, it is the intent of the Applicant to claim priority to each application that appears in the Priority Applications section of the ADS and to each application that appears in the Priority Applications section of this application.

All subject matter of the Priority Applications and the Related Applications and of any and all parent, grandparent, great-grandparent, etc. applications of the Priority Applications and the Related Applications, including any priority claims, is incorporated herein by reference to the extent such subject matter is not inconsistent herewith.

### SUMMARY

In an embodiment, epichromosomes are utilized as intranuclear delivery vehicles for various levels of addition, deletion, or modification of DNA, RNA, or protein. In an embodiment, the endogenous DNA, RNA, or protein of a subject is deleted or modified. In an embodiment, exogenous DNA, RNA, or protein is added, deleted, or modified. In an embodiment, the epichromosome delivery vehicle includes a therapeutic payload, for example, a vaccine. In an embodiment, the epichromosome delivery vehicle includes CRISPR. In an embodiment, the epichromosome includes one or more RNA recognition sequences or one or more insertion sequences.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is a partial view of an embodiment disclosed herein including an example of an epichromosomal vector with CRISPR/cas cassette.

FIG. 2A is a partial view of an embodiment disclosed herein including an example of an epichromosomal vector with CRISPR/cas cassette.

FIG. 2B is a partial view of an embodiment disclosed herein including an example of an epichromosomal vector with CRISPR/cas cassette.

FIG. 3 is a partial view of an embodiment disclosed herein including an example of an epichromosomal vector with CRISPR/cas cassette.

FIG. 4 is a partial view of the activation of CRISPR/cas.

FIG. 5 is a partial view of an embodiment disclosed herein including CRISPR/cas activation by virus invasion.

FIG. 6 is a partial view of an embodiment disclosed herein including CISPR/cas activation by auto-reactive lymphocyte(s).

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Certain embodiments include administering vectors as described herein to a host cell. In an embodiment, the host cell is located in a subject. In an embodiment, the subject is an animal or plant.

Various embodiments described herein are applicable to a number of animals, including but not limited to domesticated or wild agricultural animals, companion animals, rodents or vermin, or other domesticated or wild animals including but not limited to cow, horse, goat, sheep, goat, llama, alpaca, pig, hog, boar, bison, yak, buffalo, worm, chicken, turkey, goose, duck, fish, crab, lobster, oyster, shrimp, mussels, other shell fish, donkey, camel, mule, oxen, dog, cat, mouse, rat, hamster, rabbit, chinchilla, guinea pig, gerbil, ferret, elephant, bear, tiger, lion, dolphin, alligator, crocodile, whale, frog, toad, lizard, gecko, chameleon, raccoon, cougar, mountain lion, monkey, chimpanzee, gorilla, orangutan, ape, baboon, or other primate, giraffe, pigeon, pheasant, grouse, zebra, ostrich, bullock, water buffalo, carabao, snake, reindeer, carabiou, elk, insect, spider, antelope, deer, moose, pony, chiliquene, cormorant, parrot, parakeet, etc. or any hybrid thereof. In an embodiment, one or more gametes are modified such that hybrids, including cross-species hybrids, are generated from the fertilization. In an embodiment, the animal includes one or more reptile, amphibian, mammal, fish, or bird.

Various embodiments described herein relate to identifying and inhibiting one or more pathogens. For example, several non-limiting examples of pathogens include but are not limited to fungal, bacterial, prion, or viral pathogens. For example, pathogens include but are not limited to Streptococcus, Escherichia coli, Salmonella, Vibrio, Streptococcus, Spirilium, Shigella, Mycoplasma, yeast, or other pathogens. Sequences of many strains of parasites are available, and specific target sequences for utilization in the various embodiments disclosed herein can be adapted therefrom. These target sequences are recognized by the corresponding guide RNAs and/or Cas complexes that have been pre-programmed with the specific target recognition sequences for inactivation of the target sequences on the target.

In an embodiment, modification of the microbiome includes modifying one or more regions of the animal such that the region(s) encourage growth or sustenance of one or more non-pathogenic microorganisms including but not limited to lactobacillus, bacillus, bifidobacterium, or other non-pathogenic microorganisms. In an embodiment, modification of the microbiome includes inhibiting or destroying one or more microorganisms targeted by the CRISPR/cas vector system described herein. In this manner, a “non-pathogenic” microorganism is able to be inhibited or eliminated based on the desired medical benefits from removing or inhibiting that particular microorganism.

Various embodiments described herein are applicable to a number of plants, including but not limited to grass, fruit, vegetable, flowering trees and plants (e.g., ornamental plants, fruit plants, such as apple and cherry, etc.), grain crops (e.g., corn, soybean, alfalfa, wheat, rye, oats, barley, etc.), other food or fiber crops (e.g., canola, cotton, rice, peanut, coffee, bananas, sugar cane, melon, cucumber, sugar beet, quinoa, cassava, potato, onion, tomato, strawberry, cannabis, tobacco, etc.), or other plants (including but not limited to banana, bean, broccoli, castorbean, citrus, clover, coconut, Douglas fir, Eucalyptus, Loblolly pine, linseed, olive, palm, pea, pepper, poplar, truf, Arabidopsis thaliana, Radiata pine, rapeseed, sorghum, or Southern pine. Most of the calories consumed by humans come from members of the grass family (e.g., wheat, corn [maize], rice, oats, barley, sorghum, millet, rye, etc.), and grasses make up at least a quarter of all vegetation on Earth, rendering these important food crops worldwide. Various embodiments described herein are applicable to plant cells, seeds, pollen, fruit, zygotes, etc., as disclosed.

In certain embodiments, the vector described herein includes target sequence(s) of one or more pathogens. For example, several non-limiting examples of pathogens include but are not limited to fungal, bacterial, or viral pathogens. For example, Phakospora pachirhizi (Asian soy rust), Puccinia sorghi (corn common rust), Puccinia polysora (corn Southern rust), Fusarium oxysporum and other Fusarium spp., Alternaria spp., Penicillium spp., Pythium aphanidermatum and other Pythium spp., Rhizoctonia solani, Exserohilum turcicum (Northern corn leaf blight), Bipolaris maydis (Southern corn leaf blight), Ustilago maydis (corn smut), Fusarium graminearum (Gibberella zeae), Fusarium verticilliodes (Gibberella moniliformis), F. proliferatum (G. fujikuroi var. intermedia), F. subglutinans (G. subglutinans), Diplodia maydis, Sporisorium holci-sorghi, Colletotrichum graminicola, Setosphaeria turcica, Aureobasidium zeae, Phytophthora infestans, Phytophthora sojae, Sclerotinia sclerotiorum, Pseudomonas avenae, Pseudomonas andropogonis, Erwinia stewartii, Pseudomonas syringae pv. syringae, maize dwarf mosaic virus (MDMV), sugarcane mosaic virus (SCMV, formerly MDMV strain B), wheat streak mosaic virus (WSMV), maize chlorotic dwarf virus (MCDV), barley yellow dwarf virus (BYDV), banana bunchy top virus (BBTV), etc. See for example, U.S. Pat. No. 8,395,023, which is incorporated herein by reference.

For example, several non-limiting examples of pests capable of destroying plants include but are not limited to northern corn rootworm (Diabrotica barberi), southern corn rootworm (Diabrotica undecimpunctata), Western corn rootworm (Diabrotica virgifera), corn root aphid (Anuraphis maidiradicis), black cutworm (Agrotis ipsilon), glassy cutworm (Crymodes devastator), dingy cutworm (Feltia ducens), claybacked cutworm (Agrotis gladiaria), wireworm (Melanotus spp., Aeolus mellillus), wheat wireworm (Aeolus mancus), sand wireworm (Horistonotus uhlerii), maize billbug (Sphenophorus maidis), timothy billbug (Sphenophorus zeae), bluegrass billbug (Sphenophorus parvulus), southern corn billbug (Sphenophorus callosus), white grubs (Phyllophaga spp.), seedcorn maggot (Delia platura), grape colaspis (Colaspis brunnea), seedcorn beetle (Stenolophus lecontei), and slender seedcorn beetle (Clivinia impressifrons), as well as parasitic nematodes. Id.

For example, several non-limiting examples of target genes related to pests include but are not limited to major sperm protein, alpha tubulin, beta tubulin, vacuolar ATPase, glyceraldehyde-3-phosphate dehydrogenase, RNA polymerase II, chitin synthase, cytochromes, miRNAs, miRNA precursor molecules, miRNA promoters, etc. Id.

Certain embodiments described herein relate to epichromosomes, particularly intra-nucleus epichromosomes that persist as functioning elements for an extended time period without integrating into the host cellular genome. In an embodiment, an adeno-associated virus (AAV) vector delivered “genomic package” generates an epichromosome that persists and remains functional for an extended time period. In an embodiment, the epichromosome persists in the host cell and remains functional within the host cell for at least about 1 week, at least about 1 month, at least about 6 months, at least about 1 year, at least about 5 years, at least about 10 years, at least about 15 years, at least about 20 years, or any value therebetween. In an embodiment, the non-integrating epichromosome vector includes at least one of non-integrating adeno-associated virus vector, non-integrating Epstein Barr virus vector, non-integrating lentiviral vector, non-integrating Sendai virus vector, or any hybrid combination thereof, or the like.

In an embodiment, the epichromosome, by virtue of its lack of integrating into the host cell's genome, allows for expression of a genetic construct with less interference between it and gene expression of the host cell. Thus, in an embodiment, an epichromosome described herein includes a DNA or RNA construct that does not integrate into the host cell's chromosome(s). In an embodiment, the copy number of a construct generated from an epichromosome described herein is in excess of about 10¹⁰/kg of tissue, about 10¹¹/kg of tissue, about 10¹²/kg of tissue, or any value therebetween.

In an embodiment, an epichromosome described herein includes an RNA construct. In an embodiment, the RNA construct is transcribed within the host cell, where it is configured to target one or more pathogens (e.g., influenza, rhinoviruses, tuberculosis, etc.). In an embodiment, the RNA includes one or more of tRNA, mRNA, siRNA, microRNA, shRNA or the like.

For example, various target sequences can include viral components (e.g., viral envelope, capsid components, viral proteins or by-products, viral nucleic acids etc.), bacterial components (e.g., cell wall components, bacterial proteins, bacterial nucleic acids, bacterial by-products, etc.), yeast components (e.g., filament protein, mitochondrial protein, etc.), or inflammatory cytokines (IL-6, IL-1, IL-12, INF-alpha, etc.), or others. In an embodiment, the target sequence(s) include a DNA sequence located within the genome of the pathogen.

In an embodiment, multiple different target sequences are utilized either for the same pathogen, same type or strain of pathogen, or for different pathogens entirely. In an embodiment, the vector described herein targets at least about 1, about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, about 95, about 100, or any value therebetween sequences.

In an embodiment, the vector described herein encodes for one or more insertion sequences that are utilized for insertion or editing of a sequence. In an embodiment, the insertion sequence is inserted into the complementary target sequence before, during, after, or instead of other editing (e.g., deletion, etc.). In an embodiment, the insertion sequence is not utilized at all, but instead remains in the epichromosome.

In an embodiment, the vector(s) described herein includes one or more target sequences as part of a suite that is under control of its own promoter within the same vector as other suites. In an embodiment, multiple different suites each include their own separate promoters.

In an embodiment, the DNA or RNA encoded construct carried by the epichromosome is generated or transcribed under the control of an inducible promoter that is configured to be induced by at least one condition, including one or more of temperature, pH, pathogen, heat, magnetic field, or chemical (e.g., antibiotics). In an embodiment, the pathogen-inducible promoter recognizes at least one target pathogen antigen.

In an embodiment, a broad-ranged, multi-locus attack on one or more pathogens is permitted since multiple constructs are included in the epichromosome. For example, in an embodiment, at least 1, at least 2, at least 3, at least 4, at least 5, at least 10, at least 20, at least 30, at least 40, at least 50, at least 60, at least 70, at least 80, at least 90, at least 100, at least 125, at least 150, at least 200, at least 250, or any value therebetween different constructs are included in the epichromosome described. In an embodiment, multiple siRNAs (each having approximately two dozen bases in a typical length) upwards of several hundred different siRNAs are included in an AAV epichromosome (with a capacity on the order of 5 KB). In an embodiment, multiple different epichromosomes are provided to each of many distinct tissue-types. For example, adapting a protocol related to peptide-display library of AAV capsids with negative selection cycles for particular tissues (e.g., fibroblasts) and/or positive selection cycles for other particular tissues (e.g., mucus membranes or melanoma, etc.), an increased specificity for the positively selected tissues can be achieved. See for example, Märsch et al., Abstract Comb. Chem. High Throughput Screen November 2010; 13(9): 807-12, which is incorporated herein by reference.

In an embodiment, the epichromosome delivers at least one antigen to at least one biological tissue of a subject. In an embodiment, the epichromosome delivers at least one vaccine to at least one biological tissue of a subject. In an embodiment, the epichromosome delivers a preventative or responsive treatment to a particular disease or disorder afflicting the subject.

In an embodiment, the epichromosome is placed in a host cell (for example via infection, transfection or other form of transformation) and the transformed cell is placed in a subject. In an embodiment, the host cell is ex vivo. In an embodiment, the host cell is in vivo. In an embodiment, the host cell is in vitro. In an embodiment, the host cell is in planta. In an embodiment, the host cell is in situ. In an embodiment, the host cell originated from the same subject into which the transformed cell is placed.

In an embodiment, the subject includes at least one of a plant or animal. In an embodiment, the subject includes at least one of an amphibian, mammal, reptile, bird, or fish. In an embodiment, the subject includes a human. In an embodiment, the subject includes a food plant or ornamental plant.

In an embodiment, the treated cell originates from a tissue type including, but not limited to, blood, bone marrow, liver, brain, nerve, muscle, bone, skin, connective tissue, mucus membrane, kidney, eye, ear, mouth, spleen, gall bladder, stomach, intestinal tract, adipose, lung, heart, blood vessel, or other tissue. In an embodiment, the transformed cell remains the same cell type from which it originated. In an embodiment, the transformed cell is differentiated into another cell type different form which it originated.

In an embodiment, the epichromosome includes CRISPR (clustered, regularly interspaced short palindromic repeat) sequences. For example, in the CRISPR system, short segments of foreign DNA (spacers) are incorporated into the genome between CRISPR repeats, and serve as a “memory” of past exposures. CRISPR spacers are utilized as recognition sequences and silence exogenous genetic elements when detected. Exogenous DNA is processed by proteins encoded by some of the CRISPR-associated (cas) genes into small elements which are then inserted into the CRISPR locus near the leader sequence. RNAs from the CRISPR loci are constitutively expressed and are processed by Cas proteins to small RNAs composed of individual exogenously derived sequence elements with some flanking repeat sequence. The RNAs guide other Cas proteins to silence exogenous genetic elements at the RNA or DNA level. See for example, Makarova et al., Biol. Direct. (Abstract) 2006 Mar. 16: 1-7, which is incorporated herein by reference.

Thus, in an embodiment, the epichromosome including CRISPR is utilized for in vivo gene editing. In an embodiment, the epichromosome includes an RNA template used to identify the target edit site. In an embodiment, insertion of a new sequence is utilized following a deletion of a sequence in an editing event. In an embodiment, one or more of the target sequences include the RNA template utilized for identification of the edit site for a particular sequence editing. In an embodiment, the new inserted sequence is included in an epichromosome (either the same one as the CRISPR or a separate one). In an embodiment, the epichromosome including CRISPR is utilized to stop or correct somatic mutations. In an embodiment the epichromosome including CRISPR is utilized to detect and/or target pathogens. In an embodiment, a particular pathogen is detected and/or targeted by a pathogenic DNA sequence or other tag. In an embodiment, the epichromosome includes one or more RNA recognition sequences. In an embodiment, the epichromosome includes one or more insertion sequences. In an embodiment, the epichromosome includes at least one externally activated control sequence. In an embodiment, the externally activated control sequence includes an exogenous transcription factor necessary for operation. In an embodiment, the exogenous transcription factor includes an apoptotic inducing factor. In an embodiment, the exogenous transcription factor includes a repressor or stop codon. In this way, the epichromosomal payload is able to be controlled, as is the machinery of the transformed cell including the epichromosome.

In an embodiment, the vector(s) described herein includes one or more condition-inducible promoter. In an embodiment, the condition-inducible promoter includes at least one of a pathogen-inducible promoter, a pH-inducible promoter, a temperature-inducible promoter, a magnetic-inducible promoter, light-inducible promoter, or a chemical-inducible promoter. In an embodiment, the pathogen-inducible promoter includes at least one of PRP1/gst1 promoter, Fis1 promoter, Bet nu 1 promoter, Vst1 promoter, sesquiterpene cyclase promoter, PR-1a, Arabidopsis thaliana isolated promoter, gstA1 promoter, hsr203J promoter, str246C promoter, and sgd24 promoter, salicyclic acid-inducible promoter, ethylene-inducible promoter, thiamine-inducible promoter, benzothiadiazole-inducible promoter, pattern recognition receptor (PRRs) promoters, pathogen-associated molecular patterns (PAMPs) receptor promoters, damage-associated molecular patterns (DAMPs) receptor promoters, Toll-like receptor promoters, C-type lectin receptor promoters, mannose receptor promoters, asialoglycoprotein receptor promoters, RIG-I-like receptor promoters, or NOD like receptor promoters.

In an embodiment, the pH-inducible promoter includes at least one of a P2 promoter, P170 promoter, or FAI promoter. In an embodiment, the temperature-inducible promoter includes at least one of a promoter linked to a heat shock protein, a promoter linked to a cold shock protein, or a Tetrahymena heat inducible promoter. In an embodiment, the promoter linked to a heat shock protein includes at least one of HSP70-2 promoter, or Hvhsp17 promoter. In an embodiment, the promoter linked to a cold shock protein includes at least one of CspA promoter, CspB promoter, or CspG promoter. In an embodiment, the magnetic-inducible promoter includes at least one of magnetic nanoparticles that produce heat when exposed to an alternating magnetic field. In an embodiment, the light-inducible promoter includes at least one of carQRS promoter, or a phytochrome B/phytochrome interacting factor 3 promoter system.

In an embodiment, the chemical-inducible promoter includes at least one antibiotic-inducible promoter. In an embodiment, the antibiotic-inducible promoter includes one or more of tetracycline inducible promoter, amoxicillin-inducible promoter, tipA promoter, or LiaRS promoter system. In an embodiment, the chemical-inducible promoter includes at least one of arabinose-inducible promoter, lactate-inducible promoter, progesterone/mifepristone-inducible promoter, salinity-inducible promoter, benzoic acid-inducible promoter, steroid-inducible promoter, metallothionein promoter, cytokine-inducible promoter, or estrogen-inducible promoter.

In an embodiment, the cytokine-inducible promoter includes at least one of TNF-alpha promoter, IL-1 promoter, IL-2 promoter, IL-3 promoter, IL-4 promoter, IL-5 promoter, IL-6 promoter, IL-7 promoter, IL-8 promoter, IL-9 promoter, IL-10 promoter, IL-11 promoter, IL-12 promoter, IL-13 promoter, IL-14 promoter, IL-15 promoter, IL-16 promoter, IL-17 promoter, IL-18 promoter, IL-19 promoter, IL-20 promoter, IL-21 promoter, IL-22 promoter, IL-23 promoter, IL-24 promoter, IL-25 promoter, IL-26 promoter, IL-27 promoter, IL-28 promoter, IL-29 promoter, IL-30 promoter, IL-31 promoter, IL-32 promoter, IL-33 promoter, IL-34 promoter, IL-35 promoter, IL-36 promoter, IL-37 promoter, IL-38 promoter, GM-CSF promoter, G-CSF promoter, TNF-beta promoter, or IFN-gamma promoter.

In an embodiment, the promoter of the epichromosomal construct includes a pathogen-inducible promoter. For example, in plants pathogen-inducible promoters include PRP1 promoter (also called gst1 promoter) from potato, Fis1 promoter, Bet nu 1 promoter, Vst1 promoter, sesquiterpene cyclase promoter, PR-1a, Arabidopsis thaliana isolated promoter, gstA1 promoter, hsr203J, str246C, and sgd24. See for example, European Patent Application EP1041148; and Malnoy, et al., Planta 2003 March; 216(5):802-14 (Abstract), each of which is herein incorporated by reference. In addition, several pathogen-inducible promoters have been isolated in plants that are inducible by fungus and induce transcription of hexose oxidase, which is toxic to fungi. Furthermore, plant promoters that are pathogen-inducible by one, two, three, or more plant pathogens have been developed and can be adapted for use with various embodiments described herein. See for example, U.S. Patent App. Pub. No. 2010/0132069, which is incorporated herein by reference.

Other examples of plant pathogen-inducible promoters include those that are induced by salicyclic acid, ethylene, thiamine, or benzothiadiazole increase transcription of proteins related to targeting pathogens.

Some examples of pathogen-inducible promoters in animals include but are not limited to promoters operably coupled to receptors such as pattern recognition receptors (PRRs) that recognize pathogen-associated molecular patterns (PAMPs) which are associated with microbial pathogens or cellular stress; damage-associated molecular patterns (DAMPs) which are associated with cell damage. Other examples include Toll-like receptors, C-type lectin receptors, mannose receptors, asialoglycoprotein receptors, RIG-I-like receptors, NOD like receptors, and other PRRs found in both plants and animals.

In an embodiment, the pathogen-inducible promoter allows for highly specific and efficient induction of the payload of the epichromosome (e.g., CRISPR or another payload).

In an embodiment, the epichromosome further includes at least one toxin construct. In an embodiment, the epichromosome further includes at least one porin construct. In an embodiment, the epichromosome further includes at least one caspase construct.

Thus, in an embodiment, for example, a T cell is transformed with the epichromosome including CRISPR, and a toxin such that upon sequence recognition by CRISPR of an intracellular virus or viral component (e.g., HIV, hepatitis, tobacco mosaic virus, etc.) the virus is inactivated, and the cell itself is destroyed by the toxin. In this way, the virus is contained and not allowed to spread to nearby cells.

In an embodiment, a cell is transformed with the epichromosome including CRISPR, and a ubiquitin tag such that upon sequence recognition by CRISPR of an intracellular virus or viral component, the virus is inactivated, and the ubiquitin tag directs the invading viral complex to the proteasome.

The CRISPR system, by way of the Cas9 nucleases, can be directed by short RNAs to induce precise cleavage at endogenous genomic loci, and can edit multiple sites on the genome by allowing for coding of several sequences in a single CRISPR array. Furthermore, Cas9 can be converted into a nicking enzyme to facilitate homology directed repair. There are three CRISPR types, the most commonly used type to date is type II. For example, the CRISPR RNA targeting sequences are transcribed from DNA sequences clustered within the CRISPR array. In order to operate, the CRISPR targeting RNA is transcribed and the RNA is processed to separate the individual RNAs dependent on the presence of a trans-activating CRISPR RNA that has sequence complementarity to the CRISPR repeat (thus “guide RNA). When the trans RNA hybridizes to the CRISPR repeat, it initiates processing by the double-stranded RNA specific ribonuclease, RNAse III. So far, all identified CRISPR RNA and trans RNA guide molecules are able to bind to the Cas9 nuclease, which is activated and responds specifically to the DNA sequence complementary to the CRISPR RNA. A potential target sequence must have a specific sequence on its 3′ end, called the protospacer adjacent motif (PAM) in the DNA to be degraded but is not present in the CRISPR RNA that recognizes the target sequence. In an embodiment, the cas gene included in a vector described herein includes Cas3 or Cas9.

The CRISPR guide RNAs provide for specificity of the CRISPR-mediated nucleic acid cleavage. In addition to the naturally occurring guide RNAs, a synthetic guide RNA can be fused to a CRISPR cassette. Thus, in an embodiment, guide RNA sequences are encoded in the vector(s) described herein.

In an embodiment, an epichromosomal vector described herein is utilized to target one or more sequences that are self-antigens or antigens against which an immune response is undesirable (e.g., graft vs. host disease, auto-immune disease, allergies including anaphylactic shock, cases of sepsis, etc.) and is utilized in a lymphocyte in order to arrest antigen presentation or response to a presented antigen, or is utilized in any white blood cell in order to arrest a cytokine activation cascade. For example, the discomfort and even at times life threatening symptoms of allergies, autoimmune disease, graft vs. host disease, allergies, or sepsis, is a result of an undesirable immune response to an antigen which causes great distress in the subject.

As described herein elsewhere, in an embodiment, a Vbeta chain of a T cell receptor is targeted for inhibition or destruction by the CRISPR/cas vector disclosed by identification of that particular Vbeta as being auto-reactive (e.g., to a self-antigen, such as in the various autoimmune diseases as lupus, multiple sclerosis, rheumatoid arthritis, and others). In an embodiment, one or more components of a B cell may be targeted in a similar fashion. As described, sequences of reactive lymphocyte receptors are attained or attainable, and can be adapted for utilization with various embodiments described herein. Likewise, for known autoantigens, a target sequence for a particular auto-antigen is engineered into a CRISPR/cas vector and the auto-reactive cell is impaired such that it is unable to display the receptor that is auto-reactive. This highly specific and directed immune system regulation is beneficial in regulating particular immune responses when a high level of specificity is required. For example, in organ or tissue transplants, in auto-immune diseases or disorders, and in allergies, an inappropriate immune response that can be regulated by various embodiments disclosed herein.

In an embodiment, the vectors described herein are configured to regulate an immune response to an antigen against which an immune response is not desired, and can operate at one or more points in the immune reaction activation pathway. For example, in an embodiment, a vector described herein can operate at the point of arresting antigen processing and/or presentation in the lymphocytic cells by administering the vector to a cell of a subject, wherein the vector includes a CRISPR suite that includes one or more target sequences against which no immune response is desired, thereby arresting antigen processing and/or presentation. In an embodiment, a vector described herein can operate at the point of cytokine cascade, by administering the vector to a host cell that includes a CRISPR suite that includes one or more target sequences against cytokines or cytokine receptor activation, thereby arresting the “cytokine storm” of continued immune system activation which leads to severe trauma or even death to the subject (e.g., anaphylactic shock, sepsis, etc.). In an embodiment, the antigen against which no immune response is desired includes a transplant antigen (e.g., antigen associated with a transplanted tissue or organ, etc.), allergen (e.g., pollen, food, bee sting, animal dander, mold, dust or dust mite, etc.), or autoantigen (e.g., myelin basic protein, connective tissue components, blood vessel components, etc.), or antigen against which no immune response is desired.

In an embodiment, the vector described herein includes a CRISPR suite that includes one or more target sequences against somatic cell mutations occurring spontaneously in a cell or subject. In an embodiment, the vector described herein is utilized for surveillance of somatic cell mutations and the arrest of the initiation of cancer.

In an embodiment, administering the vector(s) described herein includes achieving internalization of the vector(s) in a host cell for example, by transformation (e.g., electroporation, calcium chloride treatment, transduction, liposomal transformation, infection etc.).

Thus, in an embodiment, an epichromosomal vector described herein is utilized to reduce or eliminate an immune response. In an embodiment, the epichromosomal vector is inserted into a T cell and is configured to arrest antigen presentation of a self-antigen or other antigen to which an immune response is not desired or tolerance of the antigen is desired. In an embodiment, the epichromosomal vector is inserted into an antigen presenting cell or a B cell in order to increase tolerance to the particular target antigen.

In an embodiment, an epichromosomal vector described herein is utilized to target one or more sequences associated with adipocyte cells, in order to regulate formation or utilization of adipocytes in a subject.

In an embodiment, an epichromosomal vector described herein is utilized to target mutations in somatic cells of a subject. As described herein, an epichromosomal vector is utilized for gene editing (e.g., insertions, deletions, etc.) as needed and is effective particularly with the CRISPR system as the payload in the epichromosomal vector.

In an embodiment, the epichromosomal vector includes means to inactivate or destroy the host cell in which the vector is contained. For example, in an embodiment, the vector encodes for one or more “suicide gene” that induces apoptosis or programmed cell death, in the host cell. For example in an embodiment the vector encodes for one or more caspases including but not limited to CASP1, CASP2, CASP3, CASP4, CASP5, CASP6, CASP7, CASP8, CASP9, or CASP10.

In an embodiment, the vector(s) described herein includes one or more externally activated control sequences. In an embodiment, one or more exogenous transcription factors or promoters are utilized in conjunction with the externally activated control sequences. In an embodiment, the one or more externally activated control sequence includes an exogenously triggered switch, either for the vector itself or for the cell that contains it.

In an embodiment, the epichromosomal vector assists in intracellular antibody-mediated degradation of a particular antigen, particularly a pathogen antigen that has been bound by IgG.

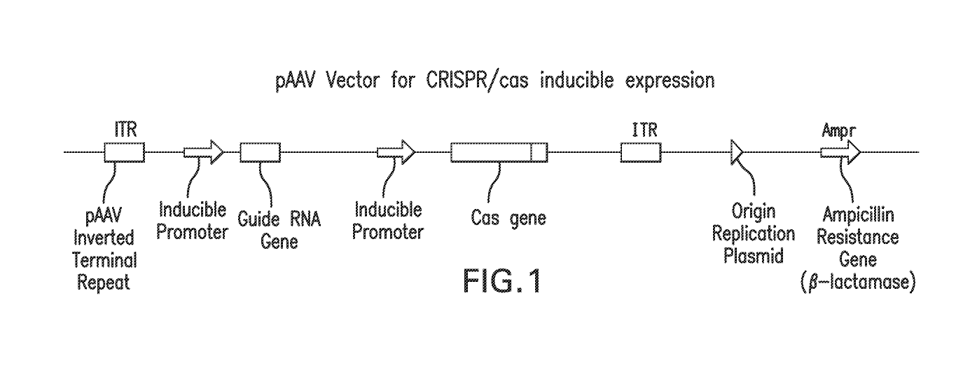

As shown in FIG. 1 a pAAV-vector with CRISPR/cas inducible expression is transduced into a cell. The vector includes an AAV inverted terminal repeat, and inducible promoter for regulating the gene encoding guide RNA, and a separate inducible promoter for regulating the cas gene, a second inverted terminal repeat, an origin of replication site, and an amp resistant gene. This is described in more detail in the Examples section herein.

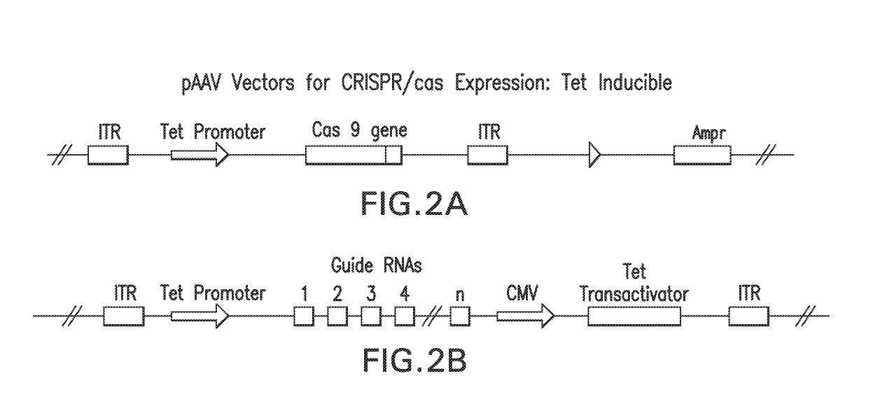

As shown in FIGS. 2A and 2B, a pAAV-vector for CRISPR/cas inducible expression includes a Tet promoter before the Cas9 gene or the guide RNAs (located on separate epichromosomal vectors), and a CMV promoter for Tet transactivator, resulting in a domino effect of activation. This is described in more detail in the Examples section herein.

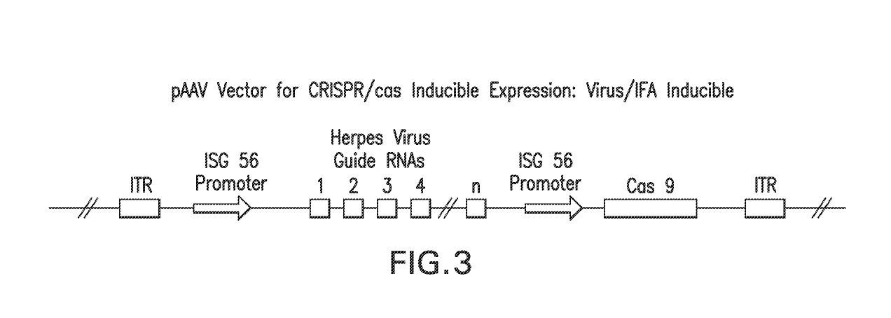

As shown in FIG. 3, the pAAV-vector for CRISPR/cas expression includes an ISG56 promoter for each of the guide RNAs and the Cas9 gene. This is described in greater detail in the Examples section herein.

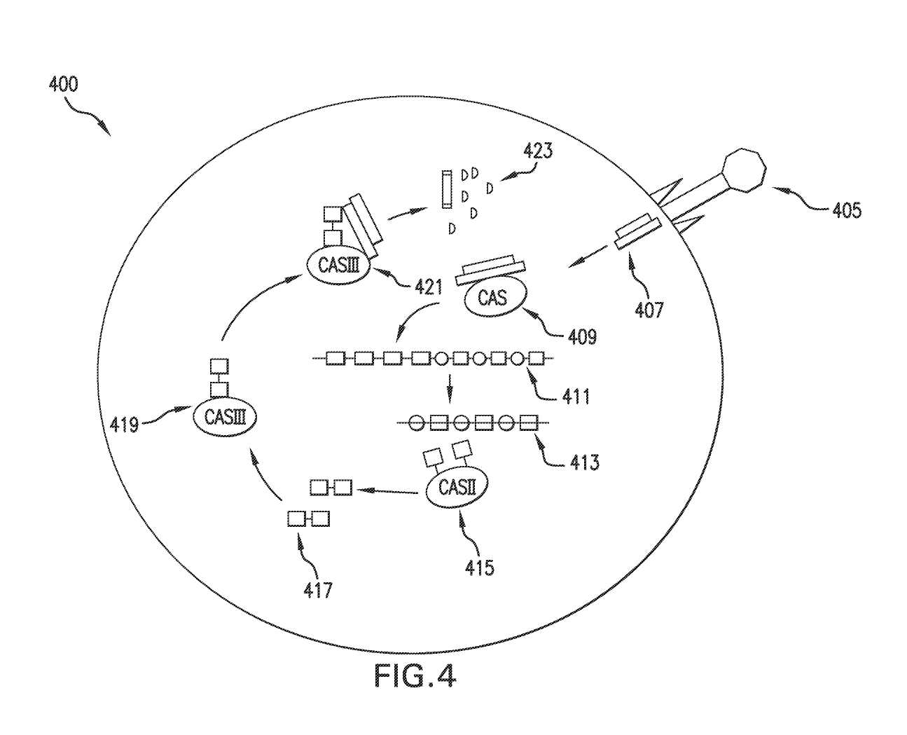

As shown in FIG. 4, the CRISPR/cas system 400 operates intracellularly by way of the cas gene 409 creating a novel spacer (target sequence) 411 that is transcribed 413 and able to be recognized by the cash complex 415 that then processes crRNAs (CRISPR RNAs) 417, that form a complex with casIII 419, and allows for targeting of the target sequence (e.g., viral DNA or autoreactive sequences, etc.) 421, and inactivation of the target 423.

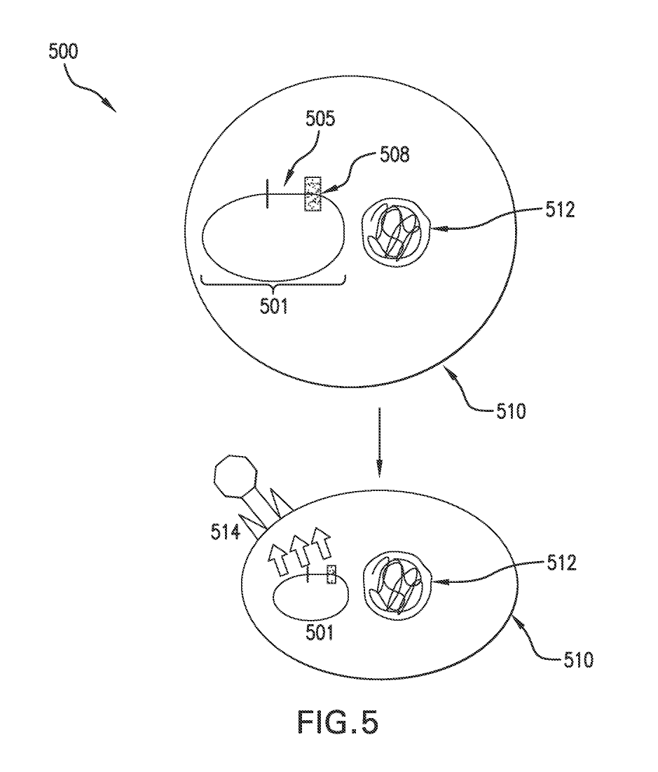

As shown in FIG. 5, an embodiment 500 in which a non-integrating epichromosome 501 with CRISPR/cas 505 cassette including a viral-inducible promoter 508 is transduced into a cell 510, where the nucleus 512 is visible. As shown, upon infection by a virus 514, the CRISPR/cas 505 cassette in the epichromosome 501 that is not integrated into the cell's genome, responds to the viral invasion by arresting or inactivating the virus.

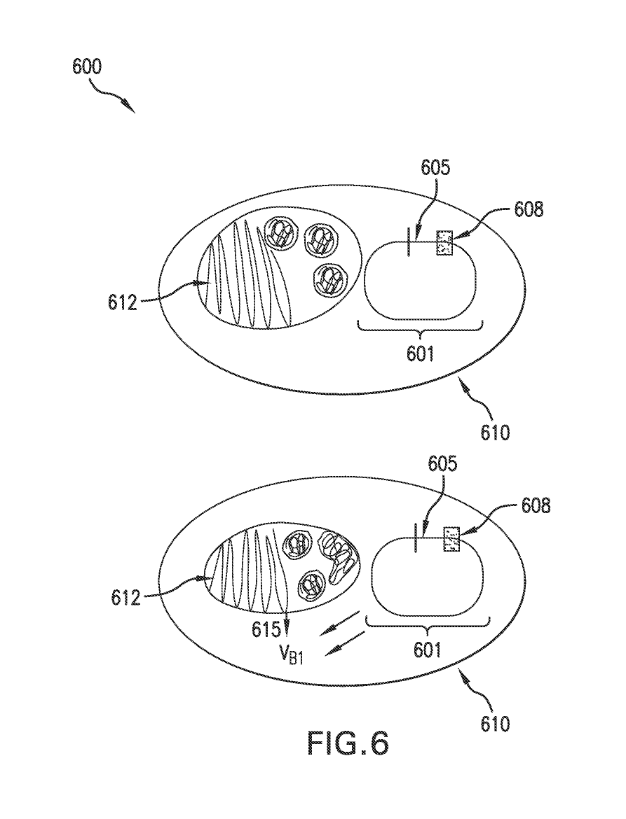

As shown in FIG. 6, an embodiment 600 in which a non-integrating epichromosome 601 with CRISPR/cas 605 cassette including an inducible promoter 608 is transduced into a T cell 610, while the nucleus is present 612 in the cell 610. In an embodiment, the T cell 610 attempts to make a Vbeta chain 615 for a T cell receptor that is auto-reactive, thereby activating the CRISPR/cas epichromosomal vector 601 and arresting T cell receptor Vbeta formation 615. In an embodiment, the T cell remains quiescent. In an embodiment, the T cell becomes anergic. In an embodiment, the T cell undergoes apoptosis.

Various non-limiting embodiments are described herein as Prophetic Examples.

### PROPHETIC EXAMPLES

### Prophetic Example 1

### An Epichromosomal Vector with a CRISPR/cas System to Ablate Hepatitis B Virus (HBV)

An adenovirus associated virus (AAV) vector is constructed to contain elements of the CRISPR/cas system (Clustered Regularly Interspaced Short Palindromic Repeats/CRISPR associated systems) which target and cleave viral DNAs. The CRISPR/cas system is delivered by an AAV vector which efficiently transduces mammalian tissues and resides long term in the cell nucleus as an epichromosome. The AAV viral vector encoding the CRISPR/cas system is derived from pAAV-MCS, a commercially available plasmid-based expression vector (e.g., see AAV Expression Vector Product Data Sheet available from Cell Biolabs, Inc., San Diego, Calif. which is incorporated herein by reference). The pAAV-MCS vector is modified by removing the constitutive CMV promoter and adding: 1) an inducible promoter, 2) a CRISPR guide RNA gene and 3) a cas gene. (See e.g., Mali et al., Science 339: 823-826, 2013 which is incorporated herein by reference.) See FIG. 1.

1) Expression of the CRISPR/cas genes in the AAV vector is controlled by an inducible promoter system (see e.g., Chen et al., Human Gene Therapy Methods 24: 270-278, 2013 which is incorporated herein by reference). A tetracycline-induced promoter system, Tet-On® 3G Inducible Expression System is available (see e.g., Tet Promoter Info Sheet from Clontech Laboratories Inc., Mountain View, Calif. which is incorporated herein by reference). The Tet promoter sequence with associated operator sequences is inserted upstream of the Cas 9 gene in the AAV vector DNA (See FIG. 2A), and a Tet-On® 3G Transactivator gene is inserted in a separate AAV vector (see FIG. 2B) under the control of a constitutive promoter, e.g., the cytomegalovirus (CMV) promoter. The Tet-On® 3G transactivator protein activates transcription from the Tet promoter when approximately 10 ng/ml of tetracycline (or doxycycline) is present. Thus oral administration of low, nontoxic amounts of tetracycline activates expression of the CRISPR/cas system, which includes the Cas 9 protein and guide RNA.

2) The design of guide RNAs with target-recognition sequences and other essential elements (e.g., hairpin and scaffold sequence) using bioinformatics methods is described (see e.g., Mali et al., Ibid.). Target DNA sequences from the Hepatitis B virus (HBV) genome are identified using bioinformatics methods and incorporated as target-recognition sequences in the guide RNAs. For example, to protect against a broad range of HBV one may select target DNA sequences from HBV genomes that are conserved among the 8 genotypes of HBV (see e.g., Norder et al., Intervirology 47: 289-309, 2004 which is incorporated herein by reference). Furthermore, variant HBV genomes with mutations in the target sequence may also be recognized and cleaved by CRISPR/cas since mutations, i.e., mismatches, that occur in the first 6 bases (i.e., 5′ end) of the selected target sequence may be recognized (see e.g., Mali et al., Ibid.) The gene(s) for one or more guide RNA(s) recognizing HBV target DNAs are expressed under the control of the Tet promoter to allow induction with tetracycline. See FIG. 2B.

3) The Cas 9 gene, which encodes a type II CRISPR/cas protein with DNAse and helicase activities is fused with a nuclear localization signal (NLS) and inserted in the AAV vector downstream from the Tet promoter sequence. For example, a human codon optimized Cas 9 gene fused to a NLS is described (see e.g., Le Cong et al., Science 339: 819-823, 2013 which is incorporated herein by reference). Expression of Cas 9 and the HBV guide RNA from separate AAV vectors is necessary to meet AAV packaging size constraints. The complete AAV CRISPR/cas vector sequences may exceed the packaging capacity of AAV (which is approximately 5 kilobases) so a modified AAV vector system may be used. For example, the AAV CRISPR/cas vector is constructed as two AAV vectors which may combine in vivo by homologous recombination. Modified AAV vectors for expression of large genes can be adapted (see e.g., Ghosh et al., Molecular Therapy 16: 124-130, 2008 which is incorporated herein by reference).

Production of viral particles with AAV CRISPR/cas genomes is accomplished by cotransfection of human embryonic kidney (HEK293) cells with an AAV CRISPR/cas vector plasmid (FIG. 2A) and helper plasmids to supply essential AAV and adenovirus gene products. Additionally, the HEK293 host cells express the adenovirus gene product, E1, which is essential for AAV particle production. Methods and cell lines for producing AAV particles with recombinant genomes can be described (see e.g., AAV Expression Vector Product Data Sheet, available from Cell Biolabs, Inc., San Diego, Calif. which is incorporated herein by reference). Cotransfection of HEK293 cells with AAV CRISPR/cas plasmid, two helper plasmids and Lipofectamine™ (available from Invitrogen, Carlsbad, Calif.) is followed by culture for 48-72 hours. The viral particles are harvested and concentrated to achieve viral genome copy numbers ranging between 10¹¹ and 10¹³ virus particles per mL (see e.g., Chen et al., Human Gene Therapy Methods 24: 270-278, 2013 which is incorporated herein by reference). The procedure is repeated to produce a second AAV CRISPR/cas vector using an AAV CRISPR/cas plasmid encoding HBV guide RNAs and the Tet transactivator protein (see FIG. 2B). The infectious titer of each AAV CRISPR/cas vector is determined (see e.g., AAV Vector Product Data Sheet, Cell Biolabs, Ibid.) and equivalent numbers of the two AAV vector particles are used for transduction of CRISPR/cas genes in vivo.

### Prophetic Example 2

### An Epichromosomal Vector Encoding a CRISPR/cas System to Treat and Prevent Herpesvirus Infections

An adenovirus associated virus (AAV) vector is constructed to contain elements of a CRISPR/cas system which target and cleave viral DNAs. The elements of a CRISPR/cas system are delivered by an AAV vector which efficiently transduces mammalian tissues and resides long term in the cell nucleus as an epichromosome. The AAV viral vector encoding the CRISPR/cas system is derived from pAAV-MCS, a commercially available, plasmid-based expression vector (e.g., see AAV Expression Vector Product Data Sheet available from Cell Biolabs, Inc., San Diego, Calif. which is incorporated herein by reference). The pAAV-MCS vector is modified by removing the constitutive CMV promoter and adding: 1) a cytokine-induced promoter, 2) genes encoding CRISPR guide RNAs and 3) a Cas 9 gene. (See e.g., Mali et al., Science 339: 823-826, 2013 which is incorporated herein by reference). See FIG. 3.

1) Expression of the CRISPR/cas genes in the AAV vector is controlled by a promoter which is induced upon viral infection. Viral infection leads to type I interferon (IFN) production by mammalian cells, and IFN stimulates a wide variety of cells to transcribe IFN-stimulated genes (ISGs). For example, ISG56 is strongly induced by type I IFNs and by viral infection (see e.g., Sen and Sarkar, Current Topics Microbiology and Immunology 316: 233-250, Springer-Verlag, Berlin 2007 which is incorporated herein by reference). The promoter for human ISG56, which contains two IFN-stimulated response elements approximately 200 bp upstream of the TATA box promoter is used to control transcription of the CRISPR/cas genes (see e.g., Fensterl and Sen, J. Interferon and Cytokine Res. 31: 71-78, 2011 and Levy et al., Proc. Natl. Acad. Sci. USA 83: 8929-8933, 1986 which are incorporated herein by reference).

2) The design of guide RNAs with target-recognition sequences and other essential elements (e.g., hairpin and scaffold sequence) using bioinformatics methods is described (see e.g., Mali et al., Ibid.). Target DNA sequences from Herpesvirus genomes are identified using bioinformatics methods and incorporated as target-recognition sequences in multiple guide RNAs. For example, target DNA sequences from the genomes of cytomegalovirus (CMV), herpes simplex virus-1 (HSV-1), herpes simplex virus-2 (HSV-2), varicella zoster (VZ), and Epstein Barr virus (EBV) are encoded in guide RNAs in a single AAV vector (see FIG. 3) and expressed in host tissues to target and cleave viral DNAs when Cas 9 protein is present. Methods and constructs to express guide RNAs can be adapted, for example, S. pyogenes guide RNAs targeting heterologous targets may be expressed in mammalian cells (see e.g., Le Cong et al, Ibid. and Deltcheva et al., Nature 471: 602-607, 2011 which is incorporated herein by reference).

3) The Cas 9 gene, which encodes a type II CRISPR/cas protein with DNAse and helicase activities is fused with a nuclear localization signal (NLS) and inserted in the AAV vector downstream from the ISG56 promoter sequence. See FIG. 3. A human codon optimized Cas 9 gene fused to a NLS is described (see e.g., Le Cong et al., Ibid.).

Production of AAV viral particles with CRISPR/cas elements under the control of the ISG56 promoter is accomplished by cotransfection of human embryonic kidney 293 (HEK293) cells with the AAV CRISPR/cas vector plasmid (FIG. 3) and helper plasmids to supply essential AAV and adenovirus gene products. See Example 1 above for details of viral particle production and determination of viral genomes/mL. Recombinant CRISPR/cas AAV particles may be tested in vitro using a mammalian cell line, e.g. HEK293 cells (available from American Type Culture Collection, Manassas, Va.). Transduction of mammalian cells with AAV vectors in vitro is described (see e.g., Le Cong et al., Ibid., and Sen et al., Scientific Reports 3: 1832, 2013; DOI: 10.1038/srep01832 which is incorporated herein by reference).

To test the transduced HEK293 cells they are infected with encephalomyocarditis virus (EMCV) to induce expression of Cas 9 and the Herpesvirus guide RNAs. EMCV infection triggers signaling through Toll-like receptors which leads to induction of transcription from the ISG56 promoter and expression of Cas 9 mRNA and the Herpesvirus guide RNAs. Following induction, the CRISPR/cas transcripts may be monitored by quantitative RT-PCR (qRT-PCR) using established methods (see e.g., Perez-Pinera et al., Nature Methods Advance Online Publication, Jul. 25, 2013; doi:10.1038/nmeth.2600 which is incorporated herein by reference). Alternatively, IFNα (available from Sigma-Aldrich, St. Louis, Mo.) may be administered to the transduced cells to induce expression of Cas 9 and the Herpesvirus guide RNAs (see e.g., Fensterl and Sen, Ibid.). Multiplex qRT-PCR with primers specific for the guide RNAs targeting CMV, HSV-1, HSV-2, VZ and EBV is used to monitor induction of each guide RNA.

To treat patients with persistent Herpesvirus infections or to prevent Herpesvirus infections subjects are administered approximately 6×10¹¹ viral genomes per kilogram of the AAV vector particles (see e.g., Nathwani et al., N. Engl J. Med. 365: 2357-2365, 2011 which is incorporated herein by reference). Induction of transcription of the CRISPR/cas elements may be by viral infection and IFN production or by administration of IFN to treat viral infection.

### Prophetic Example 3

### An Epichromosomal Vector Encoding a CRISPR/cas System to Modulate Autoreactive T Cells

An adenovirus associated virus (AAV) vector is constructed using elements of the CRISPR/cas system to modulate autoreactive T cells associated with systemic lupus erythematosus (SLE). The AAV vector is constructed with tropism for T cells and elements of the CRISPR/cas system are transcribed under the control of an inducible promoter. Autoimmune T cells associated with SLE express a finite set of T cell receptors (TCRs) comprised of selected variable region subtypes. For example TCR beta chain variable region (VB) subtypes associated with SLE include: VB2, VB8, VB11, VB14, VB16, VB19 and VB24 (see e.g., Luo et al., Clin. Exp. Immunol. 154: 316-324, 2008 and Tzifi et al., BMC Immunology 14: 33, 2013 (available online at: http://www.biomedcentral.com/1471-2172/14/33)), each of which is incorporated herein by reference). The AAV vector is constructed with an inducible promoter directing transcription of Cas 9 nuclease and CRISPR/cas guide RNAs targeting the SLE-associated VB subtype genes. Expression of the Cas 9 nuclease and VB guide RNAs results in cleavage of the corresponding SLE-associated VB genes thus disrupting expression of autoreactive TCRs and modulating autoreactive T cells.

An AAV vector to efficiently and specifically transduce T cells is selected from AAV peptide display libraries. A peptide library displayed on the capsid protein of an AAV vector is positively selected on T cells and negatively selected on non-T cells to isolate an AAV with a recombinant capsid protein that mediates efficient transduction of T cells. For example, an AAV peptide display library may be positively selected on a T cell line, e.g., Jurkat cells and negatively selected on a hepatic cell line, e.g., HepG2 (both cell lines are available from ATCC, Manassas, Va.). Methods and materials to construct AAV peptide display libraries and to select cell-specific AAV may be adapted (see e.g., Michelfelder and Trepel, Adv. Genet. 67: 29-60, 2009 and Adachi and Nakai, Gene Therapy and Regulation 5: 31-55, 2010; each of which is incorporated herein by reference). An AAV vector suitable for transducing T cells is constructed by combining pAAV-MCS, a commercially available, plasmid-based expression vector (e.g., see AAV Expression Vector Product Data Sheet available from Cell Biolabs, Inc., San Diego, Calif. which is incorporated herein by reference) with a helper plasmid encoding the recombinant capsid gene selected in vitro as described above (also see e.g., Adachi and Nakai, Ibid.).

The pAAV-MCS vector is modified by removing the constitutive CMV promoter and adding an inducible promoter (see e.g., Chen et al., Human Gene Therapy Methods 24: 270-278, 2013 which is incorporated herein by reference). A tetracycline-induced promoter system, Tet-On® 3G Inducible Expression System is available (see e.g., Tet Promoter Info Sheet from Clontech Laboratories Inc., Mountain View, Calif. which is incorporated herein by reference). The Tet-regulated promoter sequence with associated operator sequences is inserted upstream of the Cas 9 gene in the AAV vector DNA (See FIG. 2A), and a Tet-On® 3G Transactivator gene is inserted in a separate AAV vector (see FIG. 2B) under the control of a constitutive promoter, e.g., the cytomegalovirus (CMV) promoter. The Tet-On® 3G transactivator protein activates transcription from the Tet promoter when approximately 10 ng/ml of tetracycline (or doxycycline) is present. Thus oral administration of low, nontoxic amounts of tetracycline activates expression of the CRISPR/cas system.

Multiple guide RNAs targeting autoreactive VB genes are expressed in tandem under the control of the Tet promoter. Conserved sequences in the framework regions of the autoreactive VB gene subtypes are targeted. DNA sequences of the more than 50 human VB genes comprising 30 subtypes are available and able to be adapted (see e.g., Giudicelli et al., Nucleic Acids Research 33: D256-D261, 2005, which is incorporated herein by reference). Bioinformatics methods to design guide RNAs and express them in tandem can be adapted (see e.g., Le Cong et al., Ibid. and Mali et al., Ibid.) A model AAV vector with tandem guide RNA genes is shown in FIG. 2B.

Production of viral particles with AAV CRISPR/cas genomes is accomplished by cotransfection of human embryonic kidney (HEK293) cells with an AAV CRISPR/cas vector plasmid (see e.g., FIGS. 2A and 2B) and helper plasmids to supply essential AAV and adenovirus gene products. Additionally the HEK293 host cells express the adenovirus gene product, E1, which is essential for AAV particle production. See Example 1 above. Recombinant AAV particles encoding VB guide RNAs and Cas 9 are administered to SLE patients. Approximately 2-6×10¹¹ viral genomes per kilogram of each AAV vector are administered intravenously to transduce T cells. Methods and dosage for AAV vectors used in gene therapy can be adapted (see e.g., Nathwani et al., N. Engl J. Med. 365: 2357-2365, 2011, which is incorporated herein by reference). Before and after therapy with AAV vectors, the SLE patients' T cell repertoires may be monitored with next generation sequencing technology. For example, the DNA sequence of each VB gene expressed in a clinical sample and the corresponding VB subtype can be determined (see e.g., Krell et al., Haemotologica 98(9): 1388-1396, 2013 which is incorporated herein by reference).

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A composition comprising:
an non-integrating epichromosomal vector encoding at least one of a cas gene, Clustered Regularly Interspaced Short Palindromic Repeats (CRISPRs), or CRISPR guide RNA;
one or more target sequences, and
one or more condition-inducible promoters.

2. The composition of claim 1, wherein the condition-inducible promoter includes at least one of a pathogen-inducible promoter, a pH-inducible promoter, a temperature-inducible promoter, a magnetic-inducible promoter, light-inducible promoter, or a chemical-inducible promoter.

3.-13. (canceled)

14. The composition of claim 1, wherein the cas gene includes one or more of Cas3 or Cas9.

15. The composition of claim 1, wherein the non-integrating epichromosomal vector includes at least one of non-integrating adeno-associated virus vector, non-integrating Epstein Barr virus vector, non-integrating lentiviral vector, non-integrating Sendai virus vector, or any hybrid thereof.

16. The composition of claim 1, wherein multiple CRISPR guide RNA sequences are encoded in the vector.

17. The composition of claim 1, wherein the one or more target sequences include at least one of a pathogen sequence, auto-antigen, somatic cell mutation, allergen, transplant antigen, or auto-reactive lymphocyte receptor or receptor component.

18. The composition of claim 17, wherein the auto-reactive lymphocyte receptor or receptor component includes an auto-reactive variable beta chain receptor component for a T cell or a B cell.

19. The composition of claim 1, further including one or more insertion sequences encoded in the vector.

20. The composition of claim 1, wherein each of the one or more target sequences is included as part of a suite with each target sequence under control of its own promoter.

21. The composition of claim 20, wherein multiple suites of at least one target sequence are included as part of the same vector.

22. The composition of claim 1, further including one or more externally activated control sequences.

23. The composition of claim 22, wherein the one or more externally activated control sequences control transcription of one or more caspases encoded by the vector.

24. (canceled)

25. A method, comprising:
administering to a host cell,
a non-integrating epichromosome vector
encoding at least one of a cas gene, Clustered Regularly Interspaced Short Palindromic Repeats (CRISPRs), or CRISPR guide RNA;
one or more target sequences, and
a condition-inducible promoter.

26.-31. (canceled)

32. The method of claim 25, wherein the cell includes a hematapoeitic cell.

33. The method of claim 32, wherein the hematapoetic cell includes at least one of a precursor blood cell, or a differentiated blood cell.

34. The method of claim 32, wherein the hematapoetic cell includes a lymphocyte.

35. A method, comprising:
inhibiting a pathogen by administering to a host cell,
a non-integrating epichromosome vector
encoding at least one of a cas gene, Clustered Regularly Interspaced Short Palindromic Repeats (CRISPRs), or CRISPR guide RNA;
one or more target sequences of a pathogenic antigen against which an immune response is desired, and
a condition-inducible promoter.

36.-37. (canceled)

38. A method, comprising:
reducing or eliminating an immune response to an antigen against which an immune response is not desired by administering to a host cell,
a non-integrating epichromosome vector encoding at least one of a cas gene, Clustered Regularly Interspaced Short Palindromic Repeats (CRISPRs), or CRISPR guide RNA;
one or more target sequences of the antigen against which an immune response is not desired, and
a condition-inducible promoter.

39. The method of claim 38, wherein the antigen includes one or more of an auto-antigen, an allergen, or a transplant antigen.

40. A method, comprising:
in vivo genetic editing by administering to a host cell,
a non-integrating epichromosome vector encoding at least one of a cas gene, Clustered Regularly Interspaced Short Palindromic Repeats (CRISPRs), or CRISPR guide RNA;
one or more target sequences for desired editing, and
a condition-inducible promoter.

41.-42. (canceled)

43. A method, comprising:
reducing or eliminating an immune response to an antigen against which an immune response is not desired by administering to a host lymphocyte cell,
a non-integrating epichromosome vector encoding at least one of a cas gene, Clustered Regularly Interspaced Short Palindromic Repeats (CRISPRs), or CRISPR guide RNA; and
one or more target sequences of at least one lymphocyte receptor variable chain sequence against which an immune response is not desired.

44. The method of claim 43, wherein the at least one lymphocyte receptor variable chain sequence includes at least one T cell receptor variable chain beta sequence.

45. The method of claim 43, wherein the at least one T cell receptor variable chain beta sequence includes one or more sequences corresponding to auto-antigens.
